(19) United States
(12) Patent Application Publication
(10) Pub. No.: US 2006/0057215 A1
(43) Pub. Date: Mar. 16, 2006

(54) Method for the production of nanoparticles and microparticles by ternary agent concentration and temperature alteration induced immiscibility

(76) Inventors: Adrian T. Raiche, Fairport, NY (US); Joseph C. Salamone, Fairport, NY (US)

Correspondence Address: Bausch & Lomb Incorporated, One Bausch & Lomb Place, Rochester, NY, 14604-2701, US

(21) Appl. No.: 10/941,156

(22) Filed: Sep. 15, 2004

(51) Int. Cl. A61K 9/14

(52) U.S. Cl. 424/489

(57) ABSTRACT

Methods are described for forming nanoparticle- and microparticle-sized drug delivery agents. Also described are methods for incorporating one or more active therapeutic agents uniformly or non-uniformly within the particles and uses thereof.

### FIELD OF THE INVENTION

The present invention relates to a method for producing nanoparticle- and microparticle-sized spherical particles potentially consisting of or including one or more active agents. More particularly, the present invention is a method for producing nanoparticle- and microparticle-sized spherical particles useful as pure entities and as drug delivery agents, and a method for incorporating one or more active therapeutic agents uniformly or non-uniformly within the spherical particles.

### BACKGROUND OF THE INVENTION

Medication of the eyes is done commonly for two purposes—to treat the exterior of the eyes for infections such as conjunctivitis, blepharitis and keratitis sicca, and to treat the interior of eyes, i.e., intraocular treatment, for diseases such as glaucoma or uveitis. Most ocular diseases are treated through topical applications of solutions administered as eye drops. One major problem encountered with topical delivery of ophthalmic drugs is the rapid and extensive loss of drug through drainage and high tear fluid turn over. After instillation of an eye-drop in an eye, typically less than 2 to 3 percent of the applied drug penetrates the cornea. A major fraction of such instilled doses are often absorbed systemically via the conjunctiva and nasolacrimal duct. Another limitation encountered with topical delivery is a relatively impermeable corneal barrier that limits ocular absorption.

Due to inherent problems associated with the delivery of conventional ophthalmic therapeutic agents, significant effort has been directed to the development of new delivery systems such as hydrogels, nanoparticles, microparticles, liposomes and collagen shields. Ocular drug delivery is an approach to controlling and ultimately optimizing the delivery of therapeutic agents or drugs to their target tissues within the eye. Most formulation efforts to date aim to maximize ocular therapeutic agent or drug absorption by prolonging residence time on the cornea and in the conjunctival sac. Methods of prolonging such residence time include slowing the therapeutic agent or drug release rate from the delivery system and minimizing precorneal drug loss.

Many methods for the production of microspherical- and nanospherical-sized particles and methods for incorporating therapeutically active agents evenly throughout and as central cores within the microspherical and nanospherical particles for ophthalmic delivery are known. One method for producing particles in the microspherical-size range uses a solvent as a polymer or matrix sphere-forming agent that is immiscible with a bulk non-solvent. A surfactant may also be used to stabilize the emulsion formed from the immiscibility of the solvent and bulk non-solvent. Immiscibility of the solvent and non-solvent induces a lower limit on the size of the particles that form. In a static state, the solvent and non-solvent separate into two layers with the less dense layer over the more dense layer. Dispersion or emulsification of the two immiscible layers results from some form of agitation, such as ultrasonic waves, mechanical mixing or stirring, and/or vortexing. Hardened microparticle spheres are then formed by removal of the solvent by evaporation. The very small amount of solvent dissolved in the non-solvent is evaporated, and solvent contained in the stable emulsion droplets dissolves into the non-solvent to again saturate the solution.

The addition of dispersive energy competes with the immiscibility of the two solvents, acting to reduce the solvent phase droplet dimension, causing the latter to reform larger droplets. The resulting size of the microspherical particles is the balance of the two tendencies. Increasing the amount of a particular type of dispersive energy will balance the tendencies at a smaller final microspherical particle size. However, addition of dispersive energy becomes exponentially less effective; the tendency for smaller droplets to aggregate into larger ones increases exponentially as size decreases. Using an immiscible solvent/non-solvent system, it is difficult to obtain particles smaller than 500 nm in size. Because the energy spectrum used to disperse the solvent in the non-solvent is usually broad, a continuous range of size equilibriums exist. This creates a range of final particle sizes. Additionally, based on available means to introduce dispersive energy into the emulsion, the more energy that is added in an attempt to make smaller final particles, the greater the energy spectrum. Particle size distributions increase substantially as mean particle size decreases.

To produce particles smaller than 500 nm, the constraint of the tendency for droplets to aggregate is removed by using a solvent for the polymer or matrix that is miscible with a non-solvent bulk phase. Because the formation process is not dependent on the initial formation of stable emulsion droplets, surfactants can be eliminated. Variations of this method have been named nanoprecipitation and spontaneous emulsification solvent diffusion (SESD), which includes all such methods characterized by a miscible solvent/non-solvent system used with or without surfactant. Additionally, prior art also describes using a second solvent that serves as a solvent for the polymer or matrix and a second agent, but is immiscible with the non-solvent. A solution is made of the first two solvents and subsequently added to the non-solvent. This represents a combined approach where the first solvent, miscible in the bulk non-solvent, immediately diffuses from the spontaneous emulsion, but the second solvent, immiscible in the bulk non-solvent, is removed more slowly.

The advantage of methods involving some portion of a miscible solvent is the reduced capacity of aggregation, thus producing narrow size distributions of particles having a mean size less than 500 nm. The limitation with nanoprecipitation lies in the formation of a narrow size distribution of particles with a mean size from 500 nm to 1 mm in diameter. The terms “nanoprecipitation” and “spontaneous emulsification” highlight the functional aspects of these methods. It is the polymer or matrix that emulsifies in the solvent/non-solvent solution, that then precipitates on the addition of the polymer- or matrix-containing solvent to the non-solvent. The precipitation is caused by the insolubility of the polymer or matrix in the solvent/non-solvent system. Emulsification refers to the ability of the solvent to act as a plasticizer in allowing the polymer or matrix to behave as a fluid. Such emulsification enables reorganization on the same time scale as that of solvent diffusion. Hardened particles smaller than 500 nm are thus formed.

The limitation in nanoprecipitation/SESD methods arises from the practically instantaneous rate of nanoparticle formation. For most polymers, matrices and solvent systems, at mean diameters greater than 500 nm, solvent diffusion cannot occur quickly enough. The rate of hardened nanoparticle formation is too slow. As a result, and especially due to the usual lack of a surfactant, non-uniform agglomeration occurs. The resultant shapes of the hardened polymer or matrix are highly variable and dependent on, among other things, polymer or matrix/solvent droplet size upon addition to the non-solvent and polymer or matrix concentration in the solvent. Generally, porous irregular nonspherical blobs of material are formed.

A third method for formation of microparticles and nanoparticles involves using a polymer or matrix solvent solution. Microspherical or nanospherical particles are made by adding salt to the solvent solutions just described. The salt being more soluble than the polymer or matrix, causes the polymer or matrix to precipitate. The rate and amount of salt addition can be used to control the growth rate of the particles effecting great control over the breadth of the particle size distribution and on ultimate particle size. The limitation of this particular method is that a very polar solvent such as water is required to solvate the salt. Such limits the selection of polymers from which one may choose. Additionally, removal of the high salt concentration would rapidly reverse the precipitation. This “salting out” method is generally used only for polymers or matrices where reversal is strongly energetically unfavorable, such as in the case of renaturing of proteins such as bovine serum albumin (BSA). The advantage of this method is the seamless transition of a narrow distribution of particle sizes from 1 nm up to 1 mm achieved by “growing” spheres.

Colloidal carriers have also been studied for ocular drug delivery. Such colloidal carriers include mainly liposomes and nanoparticles because of their extremely small size. The main limitation of liposomes as an ocular drug delivery system are their surface charge. Positively charged liposomes seemed to be preferentially captured at the negatively charged corneal surface compared to neutral and negatively charged liposomes. Another limitation of liposomes is the instability of the lipid aggregates on the mucine surface. The vesicular aggregates of positively charged lipids are completely disintegrated on the negatively charged mucine membrane surface.

Nanoparticles as drug carriers for ocular delivery have been revealed to be more efficient than liposomes. Additionally, nanoparticles are exceptionally stable and the sustained release of drug therefrom can be relatively easily modulated. U.S. Pat. No. 5,510,103 discloses the entrapment of water-insoluble drugs in the hydrophobic core of polymeric micelles of different block copolymers. U.S. Pat. No. 5,449,513 discloses the use of various amphiphilic copolymers in the form of micelles to physically entrap water-insoluble drugs. Another patent disclosing similar subject matter includes U.S. Pat. No. 5,124,151.

Clearly, it is preferable that any ocular drug delivery system does not impair vision and reliably delivers the desired amount of therapeutic agent or drug to the targeted tissues within the eye. Therefore, the materials used to produce ocular drug delivery systems should be biocompatible, non-irritating to ocular tissues and not cause blurring or visual impairment upon use thereof.

### SUMMARY OF THE INVENTION

The present invention relates to methods for the production of nanoparticle- or microparticle-sized polymeric spherical particles for use as drug delivery agents. Polymeric spherical particles of the present invention are useful for the delivery of therapeutically effective amounts of therapeutically active agents such as but not limited to ophthalmic therapeutic agents. Polymeric spherical particles of the present invention are particularly useful in the field of ophthalmology due to the fact that the subject particles are minimally sized so as to not alter or only temporarily minimally alter visual acuity. Unaltered visual acuity during use leads to higher user compliance and greater universal appeal than traditional therapeutic treatments, which may temporarily blur vision.

The subject polymeric spherical particles are effective in the delivery of therapeutically effective amounts of therapeutically active agents. Additionally, the subject polymeric spherical particles are biocompatible and cause little or no tissue irritation.

Accordingly, it is an object of the present invention to provide a method for the production of nanoparticle- or microparticle-sized particles useful as drug delivery agents.

Another object of the present invention is to provide a method for the production of nanoparticle- or microparticle-sized drug delivery agents useful in ophthalmic applications.

Another object of the present invention is to provide a method for the production of polymeric spherical particles containing a therapeutically effective amount of a therapeutically active agent.

Another object of the present invention is to provide a method for the production of biocompatible particles for ophthalmic drug delivery.

Another object of the present invention is to provide a method for the production of biocompatible particles for ophthalmic drug delivery without or with minimal eye irritation.

Still another object of the present invention is to provide a method for the production of nanoparticle- or microparticle-sized polymeric spherical particles useful in ophthalmic applications without or with minimal visual acuity alteration.

These and other objectives and advantages of the present invention, some of which are specifically described and others that are not, will become apparent from the detailed description and claims that follow.

### BRIEF DESCRIPTION OF THE DRAWINGS

FIGURE 1 is a graph depicting the particle quantity vs. particle diameter for poly(lactic-co-glycolic) acid spheres.

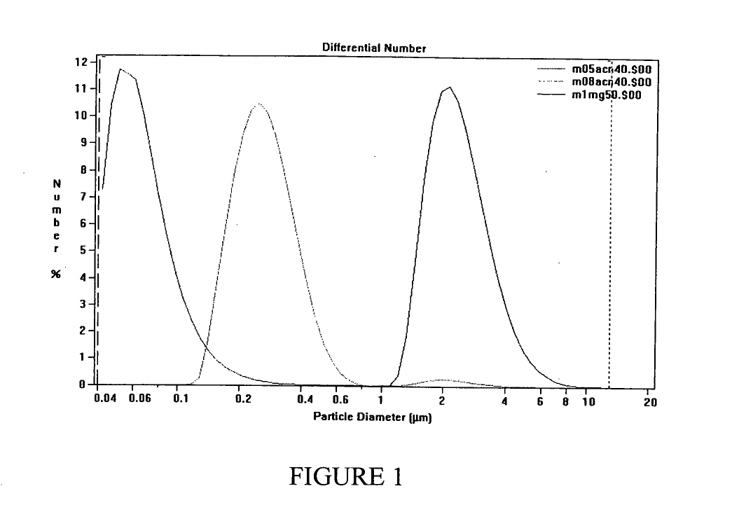

### DETAILED DESCRIPTION OF THE INVENTION

The present invention relates to methods for the production of polymeric spherical particles ranging in size from about 1 nm to about 1 mm, more preferably ranging in size from about 50 nm to about 1000 nm, and most preferably ranging in size from about 200 nm to about 400 nm. The method for producing polymeric nanoparticle- and microparticle-sized spherical particles in accordance with the present invention through ternary agent concentration and temperature alteration induced immiscibility includes several critical components. Critical components to the subject method include: 1) a solvent miscible or soluble in a non-solvent; 2) a solvent/non-solvent system in which the polymer or matrix is soluble; 3) a ternary agent soluble in the non-solvent and the solvent/non-solvent system but not soluble in the solvent; 4) a solvent having temperature dependent solubility in a solution of the non-solvent and ternary agent; and 5) a surfactant soluble in the non-solvent and solvent/non-solvent system but not soluble in the solvent. Such polymeric particles made in accordance with the present invention with therapeutically effective amounts of therapeutically active agents incorporated therein are produced using an agent solvent that: 1) is miscible or soluble in a non-solvent; 2) is not a good solvent for a ternary agent that is soluble in the non-solvent; 3) has temperature dependent solubility in a solution of the non-solvent and ternary agent; 4) is not a solvent for a surfactant that is soluble in the non-solvent; and 5) is part of a solvent/non-solvent system that is a solvent for one or more therapeutically active agents to be incorporated. The solvent for the therapeutically active agent(s) or “agent solvent” may be identical to or different than the solvent for the polymer or matrix. The solvent for the therapeutically active agent or agent solvent may or may not be a solvent for the polymer or matrix or a combination thereof.

One or more solvents may be used in accordance with the present invention. Suitable solvents for use in the method of the present invention include solvents miscible or highly soluble in a selected non-solvent such as for example but not limited to acetone, acetonitrile, ethanol, isopropyl alcohol, dimethyl sulfoxide, dimethyl formamide, tetrahydrofuran and dioxane. Preferred solvents include acetone and acetonitrile because their relatively strong solvent nature allows for particle formation of many materials. The volume of one or more solvents used in the present method is typically in the range of about 5 percent to about 50 percent.

One or more non-solvents may be used in accordance with the present invention. Suitable non-solvents for use in the method of the present invention include for example but are not limited to water, ethanol and methanol. The preferred non-solvent is water because of the ability to use secondary factors such as for example pH to further control particle formation processes. The volume of one or more non-solvents used in the present method is typically in the range of about 50 percent to about 75 percent of the solvent/non-solvent system.

Solvent/non-solvent systems of the present invention may include one or more solvents and/or one or more non-solvents. Suitable solvent/non-solvent systems for use in the method of the present invention include for example but are not limited to acetone/water and acetonitrile/water. The preferred solvent and non-solvent system is acetone/water because phase separation can be controlled through a wide range of ternary agent concentrations. The volume of solvent/non-solvent system used in the present method is typically in the range of about 10 mL to about 100 L.

One or more ternary agents may be used in accordance with the present invention. Suitable ternary agents for use in the method of the present invention include for example but are not limited to ammonium azide, ammonium bisulfite, barium acetate hydrate, barium hypophosphate, cadmium chloride, calcium acetate dihydrate, calcium chromate, calcium ethyl methyl acetate, cobalt perchlorate, iron perchlorate hexahydrate, lead chlorate hydrate, lithium hydroxide monohydrate, lithium sulfate, lithium sulfite monohydrate, potassium carbonate, potassium chloride, sodium selenate, sodium stannate (hydroxo), strontium acetate and yttrium chloride. Preferred ternary agents include sodium chloride and sodium bromide because of their strong interactions with non-solvents such as for example water, leading to solvent phase separation. The volume of one or more ternary agents used in the present method is typically in the range of about 0.1 M to about 10 M.

One or more polymers may be used in accordance with the present invention. Suitable polymers for use in the method of the present invention include for example but are not limited to polyesters, polyanhydrides, polyorthoesters, polyurethanes, polyethylene and its derivatives, all acrylate-based polymers including poly(acrylic acid), poly(methyl methacrylate) and poly(2-hydroxyethyl methacrylate), poly(N-vinylpyrrolidone) and poly(ethylenimine). Preferred polymers include polyurethanes and polysaccharides because the same allow optimal particle forming properties to be included in the material selection. The volume of one or more polymers used in the present method is typically in the range of about 0.01 percent w/v solvent/non-solvent system to about 1.0 percent w/v solvent/non-solvent system.

One or more matrices may be used in accordance with the present invention. Suitable matrices for use in the method of the present invention include for example but are not limited to trehalose, dextrose, triethanolamine, and calcium carbonate. Preferred matrices include trehalose, dextrose and triethanolamine because of their lyoprotectant and ionic interaction properties. The volume of one or more matrices used in the present method is typically in the range of about 0.01 percent w/v solvent/non-solvent system to about 1.0 percent w/v solvent/non-solvent system.

One or more solvents having temperature dependent solubility may be used in accordance with the present invention. Suitable solvents having temperature dependent solubility for use in the method of the present invention include for example but are not limited to acetone, acetonitrile, ethanol, isopropyl alcohol, dimethyl sulfoxide, dimethyl formamide, tetrahydrofuran and dioxane. Preferred solvents having temperature dependent solubility include acetone and acetonitrile because of their relatively strong solvating power. The volume of one or more solvents having temperature dependent solubility used in the present method is typically in the range of about 5.0 percent v/v of the solvent/non-solvent system to about 50 percent v/v of the solvent/non-solvent system.

One or more surfactants may be used in accordance with the present invention. Suitable surfactants for use in the method of the present invention include for example but are not limited to poly(N-vinyl pyrrolidone), poly(ethylene oxide)/poly(propylene oxide) triblock copolymers, Tweens, Sorbitans and triacyl glycerols. Preferred surfactants include poly(ethylene oxide)/poly(propylene oxide) triblock copolymers because the broad range of polymers allows for the selection of an optimal stabilizing agent. The volume of one or more surfactants used in the present method is typically in the range of about 0.1 percent w/v of the solvent/non-solvent system to about 5.0 percent w/v of the solvent/non-solvent system.

One or more agent solvents may be used in accordance with the present invention. Suitable agent solvents for use in the method of the present invention include polar charged, polar uncharged, polar, charged or neutral solvents, such as for example but not limited to chloroform, carbon tetrachloride, 1,2-dichloroethane, dichloromethane, ethyl acetate and toluene. The preferred agent solvent is ethyl acetate because of its solubility in many non-solvents. The volume of one or more agent solvents used in the present method is typically in the range of about 0.01 percent of the solvent/non-solvent system to about 10.0 percent of the solvent/non-solvent system.

One or more therapeutic agents may be used in accordance with the present invention. Suitable therapeutic agents for use in the method of the present invention include for example but are not limited to beta-blockers, anti-glaucoma agents such as for example but not limited to the beta blockers timolol maleate, betaxolol and metipranolol, mitotics such as for example but not limited to pilocarpine, acetylcholine chloride, isofluorophate, demacarium bromide, echothiophateiodide, phospholine iodide, carbachol and physostigimine, epinephrine and salts such as for example but not limited to dipivefrin hydrochloride, dichlorphenamide, acetazolamide and methazolamide, anti-cataract and anti-diabetic retinopathy agents such as for example but not limited to the aldose reductase inhibitors tolrestat, lisinopril, enalapril and statil, thiol cross-linking agents, anticancer agents such as for example but not limited to retinoic acid, methotrexate, adriamycin, bleomycin, triamcinoline, mitomycin, cisplatinum, vincristine, vinblastine, actinomycin-D, ara-c, bisantrene, activated cytoxan, melphalan, mithramycin, procarbazine and tamoxifen, immune modulators, anti-clotting agents such as for example but not limited to tissue plasminogen activator, urokinase and streptokinase, anti-tissue damage agents such as for example but not limited to superoxide dismutase, proteins and nucleic acids such as for example but not limited to mono- and poly-clonal antibodies, enzymes, protein hormones and genes, gene fragments and plasmids, steroids, particularly anti-inflammatory or anti-fibrous agents such as for example but not limited to loteprednol, etabonate, cortisone, hydrocortisone, prednisolone, prednisome, dexamethasone, progesterone-like compounds, medrysone (HMS) and fluorometholone, non-steroidal anti-inflammatory agents such as for example but not limited to ketrolac tromethamine, dichlofenac sodium and suprofen, antibiotics such as for example but not limited to loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine and tetracycline, other antipathogens including anti-viral agents such as for example but not limited to idoxuridine, trifluorouridine, vidarabine (adenine arabinoside), acyclovir (acycloguanosine), pyrimethamine, trisulfapyrimidine-2, clindamycin, nystatin, flucytosine, natamycin, and miconazole, piperazine derivatives such as for example but not limited to diethylcarbamazine, and cycloplegic and mydriatic agents such as for example but not limited to atropine, cyclogel, scopolamine, homatropine and mydriacyl.

Other therapeutically active agents or drugs include anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

Therapeutically active agents or drugs of particular interest include hydrocortisone (5-20 mcg/l as plasma level), gentamycin (6-10 mcg/ml in serum), 5-fluorouracil (−30 mg/kg body weight in serum), sorbinil, interleukin-2, phakan-a (a component of glutathione), thioloa-thiopronin, bendazac, acetylsalicylic acid, trifluorothymidine, interferon (α, β and γ), immune modulators such as for example but not limited to lymphokines and monokines and growth factors. Preferred therapeutic agents include proteins and nucleic acids because this method is relatively mild allowing high retention of biomolecule activity. The volume of one or more therapeutic agents used in the present method is typically in the range of about 1.0 percent to about 45 percent.

The present method is useful for the production of nanoparticles and microparticles through the use of ternary agent concentration and temperature alteration induced immiscibility as is described in more detail below. A solution of one or more non-solvents, one or more ternary agents, and one or more surfactants are prepared at a starting temperature. One or more polymers, one or more matrices or combinations of one or more polymers and one or more matrices are dissolved in a selected solvent or solvent system. One or more desired therapeutically active agents are dissolved in a selected agent solvent or agent solvent system. Either the polymer and/or matrix solution is mixed with the therapeutically active agent solution before addition to the non-solvent solution, or the two are added separately to the non-solvent solution. The temperature of the solution of non-solvent(s), ternary agent(s), surfactant(s), polymer and/or matrix solution and therapeutically active agent solution is either increased or decreased to reduce the solubility of the solvents in the non-solvent solution. Changes in temperature may be performed rapidly or slowly, continuously or stepwise, or linearly or non-linearly. With the associated change in temperature, solvent(s) form emulsions with the non-solvent solution. Emulsified solvents may consist of elements of the solvent system for polymer or matrix or combinations thereof, and/or elements of the solvent system for the active therapeutic agent(s). Emulsified solvent being a better solvent for polymer or matrix or a combination thereof or for one or more active agents than the solvent and non-solvent system, therapeutically active or inactive agents preferentially partition into the better solvent.

Emulsification may be controlled to preferentially force one solvent out of the non-solvent solution to effect formation of a core of material or regions with different relative amounts materials or densities of a single material, therapeutically active or inactive. Temperature alteration profile may be controlled to produce a core of material or regions with different relative amounts materials or densities of a single material, therapeutically active or inactive. Because all emulsified droplets form from the same solution and grow under similar conditions, a narrow particle size distribution can be achieved for particles from about 1 nm to about 1 mm in size.

Following or concurrent with temperature alteration is solvent removal by alteration of pressure or vapor phase composition. Solvent removal may accompany different stages of nanoparticle or microparticle formation. Removal may be controlled to remove selected solvent or solvents or part of selected solvent or solvents. The timing of temperature change and solvent removal is controlled to produce particles in the size range from 1 nm to 1 mm. In the final phase, solvent removal is extensive enough to produce hardened polymeric or matrix particles.

The method of the present invention is described in still greater detail in the following example.

### EXAMPLE 1

### Poly(lactic-co-glycolic acid) Nanospheres and Microspheres Prepared Using A Water, Acetonitrile, and Sodium Chloride System

In a specific embodiment of the method of the present invention, the non-solvent is water, the salt is sodium chloride, the surfactant is a poly(ethylene oxide)-poly(propylene oxide)-poly(ethylene oxide) triblock copolymer commercially available under the trade name Pluronic F127™ (BASF Wyandotte Corp., Wyandotte, Mich.). Acetonitrile is used as the polymer and therapeutic agent solvent. The polymer is a 50/50 copolymer of lactic and glycolic acids (PLGA) with a molecular weight of approximately 12,300. Evaluating the effect of temperature change on acetonitrile solubility in the non-solvent solution, it was determined that acetonitrile possesses a solubility maximum in a solution of sodium chloride in water at 45° C. Furthermore, it was determined that the change in solubility over the temperature range of −10° C. to 55° C., the change in the solubility was inversely proportional to the salt concentration from 5 M to 1 M. The greatest change in the solubility was for a 40 percent solution of acetonitrile in 1 M sodium chloride solution in water. At 35° C., 40 percent acetonitrile was completely soluble in the non-solvent salt solutions, but the volume occupied by the acetonitrile phase was approximately 18.6 percent of the total volume when the temperature was reduced to −10° C. After mixing the non-solvent solution, 1 M sodium chloride and 1 percent Pluronic F127™ in water, a 0.25 percent solution of PLGA was made in acetonitrile. A volume of the polymer-containing solvent solution was added to the non-solvent solution in a ratio of 4:6 by volume. The combination of the two solutions was heated in a water bath to 35° C. After reaching the temperature required to fully dissolve the solvent in the non-solvent solution, the entire volume was transferred to a vacuum flask at 10° C. and stirred. When it was confirmed that the temperature of the solvent and non-solvent solution was 10° C., vacuum was drawn to remove the acetonitrile. The experiment was repeated. However, the temperature was only decreased to 20° C. The resulting hardened PLGA particles were collected by filtration through a 100 nm filter, rinsed and dried. Analysis using dynamic light scattering, scanning electron microscopy, and atomic force microscopy confirmed that different sized particles were created.

The greater the volume of solvent emulsified in the non-solvent, the smaller the particle size. Assuming emulsion droplets originate from a continuous solution, all droplets must begin forming at the same size. Rapidly reducing the temperature induces many more emulsification events as more solvent agglomerates out of solution. For a constant amount of polymer soluble in the solvent, the more emulsion droplets that form, the smaller the mass of polymer in each droplet. After droplets are hardened, the smaller mass dissolved in the droplet results in a smaller final particle. Varying the amount of polymer dissolved in the solvent and the amount of the temperature change varies the amount of polymer that dissolves in the emulsion droplet and the number of emulsion droplets formed, respectively. Additionally, starting with a different ternary agent concentration would alter the amount of solvent that emulsifies on temperature alteration and ultimately particle size. The elements of this process provide excellent control of the formation of particles in the size range from about 1 nm to about 5 mm.

The uniqueness of the method of the present invention is that it differs from the prior art body of knowledge in that the solvent and non-solvent are miscible, a ternary agent is introduced in to the non-solvent to make the solvent and non-solvent immiscible, the concentration of the ternary agent remains constant, the ternary agent concentration makes the solvent for the polymer or matrix insoluble-not only the polymer or matrix, temperature is used to grow particles to the desired size, and a single preparation method can be used to create particles from about 1 nm to about 5 mm.

Drug delivery agents produced in accordance with the present method may be used in all cases contacting bodily fluids. Such uses include for example but not limited to topical applications, such as for example but not limited to lotions, gels or suspensions, especially for external delivery to the eye; enteric administration such as for example but not limited to direct ingestion or indirect ingestion via inhalation or naso-lacrimal duct; parenteral administration such as for example but not limited to hypodermic injection into the tissues of the body including for example but not limited to vitreous humor, aqueous humor, cornea, sclera, retina and choroids; and inhalation into the lungs.

While the invention has been described in conjunction with specific examples thereof, this is illustrative only. Accordingly, many alternatives, modifications, and variations will be apparent to those skilled in the art in the light of the foregoing description and it is, therefore, intended to embrace all such alternatives, modifications, and variations as to fall within the spirit and scope of the appended claims.

We claim:

1. A method for the production of particles comprising:
combining one or more non-solvents, one or more ternary agents and one or more surfactants to produce a non-solvent solution;
combining one or more solvents miscible with said one or more non-solvents, and one or more polymers or one or more matrices or a combination thereof to produce a solvent solution; and
combining said non-solvent solution and said solvent solution to produce particles.

2. A method for the production of particles comprising:
combining water, sodium chloride, sodium bromide, and poly(ethylene oxide)/poly(propylene oxide) triblock copolymers to produce a non-solvent solution;
combining acetone, acetonitrile and one or more polysaccharide and polyurethane polymers, or trehalose, dextrose and triethanolamine matrices, or a combination thereof to produce a solvent solution; and
combining said non-solvent solution with said solvent solution to produce particles ranging in size from about 1 nm to about 1 mm.

3. A method for the production of particles comprising:
combining water, sodium bromide or sodium chloride, and poly(ethylene oxide)/poly(propylene oxide) triblock copolymers to produce a non-solvent solution;
combining acetone or acetonitrile solvent, one or more polysaccharide or polyurethane polymers, and one or more trehalose, dextrose or triethanolamine matrices to produce a solvent solution; and
combining said non-solvent solution with said solvent solution to produce particles ranging in size from about 1 nm to about 1 mm.

4. A method for the production of a drug delivery system comprising:
combining one or more non-solvents, one or more ternary agents and one or more surfactants to produce a non-solvent solution;
combining one or more solvents and one or more polymers or one or more matrices or a combination thereof to produce a solvent solution;
combining one or more agent solvents and one or more therapeutically active agents to produce an agent solution; and
combining said non-solvent solution, said solvent solution and said agent solution to produce particles containing a therapeutically effective amount of said one or more therapeutically active agents.

5. A method for the production of a drug delivery system comprising:
combining water, sodium chloride or sodium bromide, and one or more poly(ethylene oxide)/poly(propylene oxide) triblock copolymers to produce a non-solvent solution;
combining acetone or acetonitrile solvent, one or more polyurethane or polysaccharide polymers, one or more trehalose, dextrose or triethanolamine matrices or a combination thereof to produce a solvent solution;
combining ethyl acetate and one or more therapeutically active agents to produce an agent solution; and
combining said non-solvent solution, said solvent solution and said agent solution to produce particles containing a therapeutically effective amount of said one or more therapeutically active agents.

6. A method for the production of a drug delivery system comprising:
combining water, sodium chloride, sodium bromide and one or more poly(ethylene oxide)/poly(propylene oxide) triblock copolymers to produce a non-solvent solution;
combining acetone and acetonitrile solvents, one or more polyurethane and polysaccharide polymers, trehalose, dextrose and triethanol amine matrices or a combination thereof to produce a solvent solution;
combining ethyl acetate and one or more therapeutically active agents to produce an agent solution; and
combining said non-solvent solution, said solvent solution and said agent solution to produce particles containing a therapeutically effective amount of said one or more therapeutically active agents.

7. A method for the production of a drug delivery system comprising:
combining one or more non-solvents, one or more ternary agents and one or more surfactants to produce a non-solvent solution;
combining one or more solvents, one or more therapeutically active agents and one or more polymers or one or more matrices or a combination thereof to produce a solvent solution; and
combining said non-solvent solution and said solvent solution to produce particles containing a therapeutically effective amount of said one or more therapeutically active agents.

8. A method for the production of a drug delivery system comprising:
combining water, sodium chloride or sodium bromide, and one or more poly(ethylene oxide)/poly(propylene oxide) triblock copolymers to produce a non-solvent solution;
combining acetone or acetonitrile solvent, one or more therapeutically active agents and one or more polymers or one or more matrices or a combination thereof to produce a solvent solution; and
combining said non-solvent solution and said solvent solution to produce particles containing a therapeutically effective amount of said one or more therapeutically active agents.

9. A method for the production of a drug delivery system comprising:
combining water, sodium chloride, sodium bromide and one or more poly(ethylene oxide)/poly(propylene oxide) triblock copolymers to produce a non-solvent solution;
combining acetone and acetonitrile solvent, one or more therapeutically active agents and one or more polymers or one or more matrices or a combination thereof to produce a solvent solution; and
combining said non-solvent solution and said solvent solution to produce particles containing a therapeutically effective amount of said one or more therapeutically active agents.

10. A method for the production of particles comprising:
combining one or more solvents and one or more non-solvents that are miscible;
introducing one or more ternary agents into said one or more non-solvents to make said one or more solvents and said one or more non-solvents immiscible, with said one or more ternary agents making one or more polymers or one or more matrices and optionally one or more therapeutically active agents in said one or more solvents insoluble; and
varying temperature to control particle formation to a desired size.

11. A method for the production of particles comprising:
combining acetone or acetonitrile solvent with water;
introducing sodium chloride or sodium bromide to make said acetone or acetonitrile solvent and said water immiscible, with said ternary agent making one or more polymers or one or more matrices and optionally one or more therapeutically active agents in said acetone or acetonitrile solvent insoluble; and
varying temperature to control particle formation to a desired size.

12. A method for the production of particles comprising:
combining acetone and acetonitrile solvents with water;
introducing sodium chloride and sodium bromide into to make said acetone and acetonitrile solvents and said water immiscible, with said ternary agents making one or more polymers or one or more matrices and optionally one or more therapeutically active agents in said acetone and acetonitrile solvents insoluble; and
varying temperature to control particle formation to a desired size.

13. The method of claim 1, 2, 3, 4, 5, 6, 7, 8 or 9 wherein size of said particle controlled through temperature variation.

14. The method of claim 1, 2, 3, 4, 5, 6, 7, 8, 9, 10, 11 or 12 wherein said particles are about 1 mm to about 1 nm in size.

15. The method of claim 1, 2, 3, 4, 5, 6, 7, 8, 9, 10, 11 or 12 wherein said particles are about 50 nm to about 1000 nm in size.

16. The method of claim 1, 2, 3, 4, 5, 6, 7, 8, 9, 10, 11 or 12 wherein said particles are about 200 nm to about 400 nm in size.

17. The method of claim 1, 4, 7 or 10 wherein said one or more solvents are selected from the group consisting of acetone, acetonitrile, ethanol, isopropyl alcohol, dimethyl sulfoxide, dimethyl formamide, tetrahydrofuran and dioxane.

18. The method of claim 4 wherein said one or more agent solvents are selected from the group consisting of chloroform, carbon tetrachloride, 1,2-dichloroethane, dichloromethane, ethyl acetate and toluene.

19. The method of claim 1, 4, 7 or 10 wherein one of said one or more solvents is acetone, acetonitrile or a combination thereof.

20. The method of claim 4 wherein one or said one or more agent solvents is ethyl acetate.

21. The method of claim 1, 4, 7 or 10 wherein said one or more non-solvents are selected from the group consisting of water, alcohols, ethers, amine-containing solvents, carboxyl-containing solvents and organic solvents.

22. The method of claim 1, 4, 7 or 10 wherein one of said one or more non-solvents is water, methanol, ethanol or a combination thereof.

23. The method of claim 1, 4, 7 or 10 wherein said one or more ternary agents are selected from the group consisting of ammonium azide, ammonium bisulfite, barium acetate hydrate, barium hypophosphate, cadmium chloride, calcium acetate dihydrate, calcium chromate, calcium ethyl methyl acetate, cobalt perchlorate, iron perchlorate hexahydrate, lead chlorate hydrate, lithium hydroxide monohydrate, lithium sulfate, lithium sulfite monohydrate, potassium carbonate, potassium chloride, sodium selenate, sodium stannate (hydroxo), strontium acetate and yttrium chloride.

24. The method of claim 1, 4, 7 or 10 wherein one of said one or more ternary agents is sodium chloride, sodium bromide or a combination thereof.

25. The method of claim 1, 4, 7 or 10 wherein said one or more polymers are selected from the group consisting of polyesters, polyanhydrides, polyorthoesters, polyurethanes, polyethylene and its derivatives, all acrylate-based polymers including poly(acrylic acid), poly(methyl methacrylate) and poly(2-hydroxyethyl methacrylate), poly(N-vinylpyrrolidone) and polyethylenimine.

26. The method of claim 1, 4, 7 or 10 wherein said one or more matrices are selected from the group consisting of trehalose, dextrose, triethanolamine, and calcium carbonate.

27. The method of claim 1, 4, 7 or 10 wherein said one or more surfactants are selected from the group consisting of poly(N-vinylpyrrolidone), poly(ethylene oxide)/poly(propylene oxide) triblock copolymers, Tweens, Sorbitans and triacyl glycerols.

28. The method of claim 4, 5, 6, 7, 8, 9, 10, 11 or 12 wherein said one or more therapeutically active agents are selected from the group consisting of beta-blockers, anti-glaucoma agents, anti-cataract agents, anti-diabetic retinopathy agents, anti-cancer agents, anti-clotting agents, anti-tissue damage agents, proteins, nucleic acids, steroids, non-steroidal anti-inflammatory agents, antibiotics, anti-pathogens, anti-viral agents, cycloplegic agents, mydriatic agents, anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

29. The method of claim 1, 2, 3, 4, 5, 6, 7, 8, 9, 10, 11 or 12 wherein said particles are useful as drug delivery agents.

30. The method of claim 1, 2, 3, 4, 5, 6, 7, 8, 9, 10, 11 or 12 wherein said particles are useful as ophthalmic drug delivery agents.

31. An improved method for producing spherical particles by combining a non-solvent solution and a solvent solution wherein the improvement comprises producing said particles through ternary agent concentration and temperature alteration induced immiscibility.

32. The method of claim 31 wherein said particles further include one or more therapeutically effective agent.

33. The method of claim 31 or 32 wherein said particles are about 1 mm to about 1 nm in size.

34. The method of claim 31 or 32 wherein said particles are about 50 nm to about 1000 nm in size.

35. The method of claim 31 or 32 wherein said particles are about 200 nm to about 400 nm in size.

36. A method of using particles produced through the method of claim 1, 4, 7, 10 or 33 comprising:
topically applying said particles on a patient in the form of a lotion, gel or suspension.

37. A method of using particles produced through the method of claim 1, 4, 7, 10 or 33 comprising:
enterically administrating said particles to a patient through direct ingestion or through indirect ingestion.

38. A method of using particles produced through the method of claim 1, 4, 7, 10 or 33 comprising:
parenterally administrating said particles to a patient.

39. A method of using particles produced through the method of claim 1, 4, 7, 10 or 33 comprising:
administering said particles to a patient through inhalation of said particles.
